(19) United States
(12) Patent Application Publication
(10) Pub. No.: US 2003/0109045 A1
(43) Pub. Date: Jun. 12, 2003

(54) RNA silencing suppression

(76) Inventors: Richard S. Nelson, Ardmore, OK (US); Xin Shun Ding, Ardmore, OK (US)

Correspondence Address: Robert E. Hanson, FULBRIGHT & JAWORSKI L.L.P., SUITE 2400, 600 CONGRESS AVENUE, AUSTIN, TX, 78701-3271, US

(21) Appl. No.: 10/223,070

(22) Filed: Aug. 16, 2002

Related U.S. Application Data
(60) Provisional application No. 60/313,185, filed on Aug. 17, 2001.

(51) Int. Cl. A01H 1/00; C12N 15/82

(52) U.S. Cl. 435/468; 800/280

(57) ABSTRACT

The invention provides methods of suppressing gene silencing and/or stabilizing expression of a coding sequence in a cell comprising expressing a 126 kDa protein and/or the 183 kDa protein of a subgroup sindbis plant virus in the cell. The invention can thus be used to avoid the deleterious effects of gene silencing in a cell. Also provided by the invention are methods of delivering a polypeptide of interest to a limited part of a plant.

This application claims the priority of U.S. Provisional Patent Application Serial No. 60/313,185, filed Aug. 17, 2001, the entire disclosure of which is specifically incorporated herein by reference.

### BACKGROUND OF THE INVENTION

1. Field of the Invention

The current invention relates generally to the field of molecular biology. More particularly, it concerns methods for modulating gene expression.

2. Description of Related Art

RNA silencing is a phenomenon that can affect systemic virus accumulation. It was first identified in plants wherein aberrant or overexpressed RNA sequences are targeted for destruction (reviewed in Vance and Vaucheret, 2001). The destruction of the RNA is sequence-specific and proceeds through the synthesis of small (21-25 nucleotides) RNAs of both sense and antisense conformation (Hamilton and Baulcombe 1999, Zamore et. al, 2000). RNA silencing has been shown to be a viral defense mechanism in plants (reviewed in Vance and Vaucheret, 2001; Carrington et al. 2001; Baulcombe 1999). RNA silencing in the host prevents virus accumulation in systemic leaves of virus-challenged, nontransgenic plants (Al-Kaff et al., 1998; Ratcliff et al., 1997; Covey, et al. 1997). Virus challenge also leads to silencing of transgenes expressing homologous viral RNA sequences in systemic tissue of transgenic plants (e.g. Al-Kaff et al., 1998). Previously, it has been shown that RNA silencing can also target challenge virus RNA if the transgene contains viral sequences (Lindbo et al., 1993; Smith et al., 1994).

Plant viruses contain sequences that can suppress RNA silencing in the infected host (e.g. Voinnet et al. 1999). In some instances, specific viral proteins capable of suppressing RNA silencing in transgenic plants have been identified (Anandalakshmi et al., 1998; Brigneti et al., 1998; Kasschau and Carrington, 1998; Voinnet et al. 1999 and 2000). These proteins were previously shown to be necessary for the local or phloem-dependent accumulation of their encoding viruses in their respective hosts (Cronin et al., 1995; Ding et al., 1995a; Hong et al. 1997; Bonneau et al. 1998; Scholthof et al. 1995). All of the proteins are non-structural (i.e. do not form the capsid of the virus) and their functions in virus accumulation and suppression of RNA silencing are not fully understood.

The helper component-protease (HC-Pro) from potyviruses promotes replication and accumulation of the virus, at least partially through its proteinase activity, and is necessary for aphid transmission (reviewed in Revers et al., 1999). HC-Pro has been shown to inactivate pre-existing RNA silencing (Brigneti et al. 1998). The decline of RNA silencing is also associated with a decrease in the accumulation of the small RNAs associated with this phenomenon (Llave et al. 2000, Mallory et al. 2001). The 2b protein, which is not present in the related bromoviruses, is a virulence determinant (Ding et al., 1996a). The 2b gene or its protein is not necessary for virus replication or cell-to-cell spread (Ding et al., 1995a). The protein does not inhibit RNA silencing in already silenced tissue, but inhibits silencing from occurring in newly developing tissue (Brigneti et al. 1998). The 2b protein localizes to the nuclei of tobacco cells and this function is necessary for efficient suppression of RNA silencing (Lucy et al. 2000). Recently, it was shown that the 2b protein inhibits salicylic acid-mediated virus resistance (Ji and Ding, 2001). The p25 protein encoded by Potato virus X recently was determined to block the systemic spread of the RNA silencing signal in plants (Voinnet et al. 2000). Further research is necessary to determine with what host or viral proteins these factors interact to prevent silencing from occurring.

The method by which other viral proteins, such as the AC2, P1 and 19 kDa proteins from African cassaya virus, Rice yellow mottle virus and Tomato bushy stunt virus, respectively, suppress silencing has not been determined (Voinnet et al. 1999). Other non-structural viral proteins or their open reading frames (orfs) have been shown to affect phloem-dependent accumulation of their respective viruses (for review see Nelson and van Bel, 1998). These include the αa protein of barley stripe mosaic virus, 1a and 2a proteins of Brome mosaic virus and CMV, 129 and/or 186 kDa protein(s) of SHMV, and 126 and/or 183 kDa protein(s) of TMV (De Jong and Ahlquist, 1995; Deom et al., 1997; Lakshman and Gonsalves, 1985; Nelson et al., 1993; Roossinck and Palukaitis, 1990; Traynor et al., 1991; Weiland and Edwards, 1994). Although these proteins are known to affect virus accumulation, their potential to suppress RNA silencing has not been investigated.

The basis for the different disease symptoms elicited by two strains of TMV on Nicotiana tabacum L. cv. Xanthi have been previously characterized. The attenuated “masked” strain (M, Holmes, 1934) causes a very mild chlorotic mottling of systemically-infected leaves while the severe U1 strain causes a bumpiness (i.e. rugosity), likely due to nonuniform cell expansion or division in the cells of the leaf lamina, and a well-defined light-green, dark-green mosaic on systemically-infected leaves (e.g. Shintaku et al., 1996). The portion of the viral genome responsible for the different symptoms induced by these viruses is within the 126 kDa protein orf (Holt et al., 1990). Through mutagenesis of cDNA clones of the masked (M^{IC}) and U1 strains it was determined that eight nucleotides in the 126 kDa protein orf, resulting in eight amino acid differences in the 126 kDa and 183 kDa proteins of these viruses, controlled the symptom and phloem-dependent accumulation phenotypes of the infectious transcripts (Derrick et al., 1997; Shintaku et al., 1996). Further substitutions in specific codons and analysis of infections caused by virus containing these altered sequences led to the conclusion that the 126 kDa and/or 183 kDa protein, and not the viral RNA, is responsible for the symptom phenotypes displayed by the viruses (Bao et al., 1996). M^{IC}1,3 (FIG. 1) does not induce systemic symptoms on leaves of N. tabacum, although it accumulates in the inoculated leaves (Shintaku et al., 1996).

Viruses are believed to cause yield reductions in plants through their movement and accumulation in tissue distant from the initially inoculated site (Matthews, 1991). As such, it is important to understand the method by which viruses accumulate in these distant tissues (i.e. how viruses accumulate systemically). Identifying viral and host factors that control systemic virus accumulation and the location where they function will aid in designing future generations of transgenic plants which maintain yields in spite of virus challenge. In addition, understanding how viruses accumulate and move through the host will provide clues to how host macromolecules accumulate and move through plants. Therefore, there is a great need in the art for further understanding of the physiological interaction between plant viruses and plants. Lastly, there is need in the art for methods of enhancing accumulation of foreign proteins in plants being used as factories for protein production.

### SUMMARY OF THE INVENTION

In one aspect, the invention provides a method of suppressing gene silencing and/or stabilizing expression of a coding sequence in a cell comprising expressing a 126 kDa protein and/or the 183 kDa protein of a Sindbis-like plant virus in the cell. For the purposes of this submission, only plant sindbis-like viruses that have a methyltransferase domain upstream of a helicase domain with no intervening known protease domain, all part of one protein, are encompassed with within the definition of “Sindbis-like plant virus.” This includes viruses in the following families or genuses as defined by Hull (2002): Tobamoviruses, Tobraviruses, Hordeiviruses, Bromoviridae, Benyviruses, Idaeoviruses, Potexviruses, Allexiviruses, Foveaviruses, Pomoviruses, Caralviruses and Vitiviruses. These are defined as “subgroup sindbis” hereafter.

Thus, the method may also comprise infecting the cell with a subgroup sindbis plant virus encoding the 126 kDa protein and/or the 183 kDa protein, or their homologues, and allowing the 126 kDa protein and/or the 183 kDa protein, or their homologues, to be expressed. Such a coding sequence may be introduced into the genome of the cell or a progenitor thereof by genetic transformation and also may be present in more than one copy in the cell. In one embodiment of the invention, comprises infecting the cell with a subgroup sindbis plant virus encoding the 126 kDa protein and/or the 183 kDa protein, or their homologues, and allowing the 126 kDa protein and/or the 183 kDa protein, or their homologues, to be expressed. Such a coding sequence may be introduced into the genome of the cell or a progenitor thereof by genetic transformation and also may be present in more than one copy in the cell.

In the claimed method, expressing may comprise transforming the cell or a progenitor thereof with a nucleic acid sequence encoding the 126 kDa protein and/or the 183 kDa protein. In certain further embodiments of the invention, the subgroup sindbis plant virus is selected from the group consisting of Tobamoviruses, Tobraviruses, Hordeiviruses, Bromoviridae, Benyviruses, Idaeoviruses, Potexviruses, Allexiviruses, Foveaviruses, Pomoviruses, Carlaviruses or Vitiviruses.

In a further embodiment of the invention, the coding sequence may be expressed from the plant's genome and/or the virus may comprise and express the coding sequence. The nucleic acid sequence encoding the 126 kDa protein and/or the 183 kDa protein may or may not be fused to the coding sequence. The cell may be a plant cell. The cell may also further be comprised in a plant. In one embodiment of the invention, the plant is a dicotyledonous plant. Examples of dicotyledonous plants include tobacco, tomato, potato, soybean, cotton, canola, alfalfa, sunflower, and cotton. In certain embodiments of the invention, the plant is selected from the group consisting of Nicotiana tabacum and Nicotiana benthamiana. The plant may also be a monocotyledonous plant. Examples of monocotyledonous plants include wheat, maize, rye, rice, oat, barley, turfgrass, sorghum, millet and sugarcane.

In another aspect, the invention provides a method of delivering a polypeptide of interest to a limited part of a plant comprising the step of infecting a plant with a Sindbis-like plant virus, wherein the virus encodes a 126 kDa protein and/or 183 kDa protein. In one embodiment of the invention, one or more mutations are at position 598-601 of the 126 kDa and/or 183 kDa protein. In certain embodiments of the invention, the amino acid at position 598 is not methionine; the amino acid at position 598 is arginine; the amino acid at position 601 is not lysine; and/or the amino acid at position 601 is glutamic acid.

In another embodiment of the invention, the plant is a dicotyledonous plant. Examples of dicotyledonous plants include tobacco, tomato, potato, soybean, cotton, canola, alfalfa, sunflower, and cotton. In certain embodiments of the invention, the plant is selected from the group consisting of Nicotiana tabacum and Nicotiana benthamiana. The plant may also be a monocotyledonous plant. Examples of monocotyledonous plants include wheat, maize, rye, rice, oat, barley, turfgrass, sorghum, millet and sugarcane. In the method, the subgroup sindbisplant virus may be selected from the group consisting of Tobamoviruses, Tobraviruses, Hordeiviruses, Bromoviridae, Benyviruses, Idaeoviruses, Potexviruses, Allexiviruses, Foveaviruses, Pomoviruses, Caralviruses and Vitiviruses.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1 Genome organization of TMV showing the location of amino acid differences between M^{IC} and M^{IC}1,3. Open reading frames (orfs) are indicated by bars. Nontranslated regions are denoted as lines. UAG designates the leaky amber termination codon. 126 and 183 kDa proteins function in virus replication and spread of Tobacco mosaic virus. The movement protein (MP) functions in cell-to-cell spread. The coat protein encapsidates the viral RNA and also functions in vascular-dependent accumulation. The shaded area in 126 kDa protein orf indicates the methyltransferase (MT) domain of this protein. The cross-hatched area in 126 kDa orf indicates the helicase domain of this protein. The area in 126 kDa orf between nt 1323 and 1430 indicates a region of conservation within Tobamoviruses, Bromoviridae, Tobraviruses, Ilarviruses and Hordeiviruses with no known function. Domains I and II are regions with less sequence similarity to subgroup sindbis viruses and no known function (Shintaku et al. 1996). Numbers 1-8 indicate the position of the 8 amino acids that differ between M^{IC} and the severe U1 strain of TMV. These amino acids are responsible for the different symptoms induced by these two strains. Sequence differences between M^{IC} and M^{IC}1,3 126 and 183 kDa proteins are shown along with the amino acid location where they differ.

FIG. 2 Immunoblot detection of viral movement protein in TMV-infected protoplasts 7 hours post-inoculation with M^{IC} U1 or M^{IC}1,3 (denoted 1,3 in figure). H=healthy (i.e. uninfected tissue)

FIG. 3 Accumulation of viral RNA in inoculated and systemic tissue after inoculation with M^{IC} 1,3. A percentage of plants (20%) inoculated with M^{IC} 1,3 were found to display systemic symptoms after time, with chlorotic areas observed in leaves interspersed with dark green areas (panel A). Leaves from these systemic leaves were harvested and progeny virus sequenced using specific primers (arrows below viral genome indicate sequenced regions in panel B). A single nucleotide substitution resulting in a single amino acid substitution was found to cause the altered accumulation pattern (mutant referred to as M^{IC}1,3,6*=M^{IC}1,3,1864 or 1,3,6*). A second virus was identified in systemic tissue of another inoculated plant that also had a single substitution in this area (mutant referred to as M^{IC}1,3,6 or M^{IC} 1,3,1872). M^{IC}1,3,1872 induced symptoms identical to M^{IC}1,3,6*.

FIG. 4 M^{IC}1,3 accumulation in systemic tissue is host dependent. M^{IC}1,3 accumulates in systemic tissue of N. benthamiana (panel A), although the symptoms induced are less than those induced by the second site mutant, M^{IC}1,3,6* (panel B) at 16 days post inoculation.

FIG. 5 M^{IC}1,3 is able to enter the vascular tissue and move systemically in N. tabacum cv. Xanthi. Grafting studies were conducted in which reciprocal grafts were made between N. tabacum and N. benthamiana rootstocks and scions. Accumulation of virus was determined at 8 days post inoculation via ELISA with antibodies against the CP of TMV. Inoculated leaves or shoot apices containing the youngest mature leaf and younger were harvested for analysis. Values for the grafts with N. benthamiana as the rootstock are means +/− the standard deviation for 2 replicates. Values for the grafts with N. tabacum as the rootstock are values from individual samples from an experiment. These results indicate that M^{IC}1,3 could enter and move through the vascular tissue of the nonsupportive host (N. tabacum), but either could not exit or establish a systemic infection in the nonsupportive host.

FIG. 6 Delay of transgene silencing maps to the 126/183 kDa proteins of TMV as shown by inoculation with various strains and mutants of TMV. Transgenic plants expressing the 126 kDa protein fused to GFP were inoculated with viruses M^{IC}1,3; M^{IC}; and M^{IC}1-8; (M^{IC}1-8 has an equivalent phenotype to the U1 strain) or mock-inoculated with buffer only (mock). Images were taken of systemic leaves, approximately 7 cm in midrib length, at various days post inoculation. At 4 and 7 days post inoculation the images were taken of the leaf surface and of a transverse section of the midrib. At 13 and 18 days post inoculation images were taken of the leaf surface. Boxed images were enhanced equally to better show GFP expression. Images were obtained using a confocal system (model 1024ES, BioRad) attached to an upright microscope (Zeiss Axioskop) using previously described filters (Cheng et al. 2000). The results indicated that M^{IC}1,3 induced silencing of vein-associated systemic tissue by 7 days post inoculation, while M^{IC} and M^{IC}1-8 comparatively delayed silencing for varying periods of time. In particular, it was found that GFP silencing was veincentric and that the observed delay in silencing maps to the 126 kDa and/or 183 kDa protein of TMV This suggests that M^{IC} and M^{IC} 1-8 are able to suppress silencing of this tissue.

FIG. 7A, B Small RNAs. hallmarks of the presence of RNA silencing, are present in systemic leaves of plants undergoing silencing for GFP expression at 13 dpi. FIG. 7A: Tissue analyzed is from leaf one above those shown in FIG. 6. Identification of small RNAs was performed by northern-blot, using the 126 kDa protein orf as a probe, as described (Itaya et al., 2001). Numbers below are given in arbitrary units corresponding to relative radioactivity of equal areas of membrane corresponding to position of bands. ssDNA is 25-mer oligonucleotide stained with ethidium bromide in 15% urea-PAGE. The presence of small RNAs in these systemic leaves indicates that post transcriptional gene silencing is occurring in all virus-inoculated samples. The amount of small RNAs positively correlates with virus-stabilized expression of GFP. FIG. 7B: RNAs of molecular weight equivalent to 500-200 nucleotides stained with ethidium bromide are present in the same samples as shown in Panel 7A.

FIG. 8 Suppression of in trans transgene silencing maps to amino acid position 601 of the 126/183 kDa protein(s). Leaves of transgenic plants expressing GFP (line 16C) were infiltrated with Agrobacterium tumefaciens expressing the same GFP gene or mock infiltrated with buffer only at the 3-5 leaf stage. Plants silenced for GFP were challenged in newly and fully silenced leaves with virus or mock inoculated (1,3,6; 1,3; 6 and 1-8=TMV mutants M^{IC}1,3,6; M^{IC}1,3; M^{IC}6 and M^{IC}1-8 (see Shintaku et al. 1996 for further description), CMV=Cucumber mosaic virus, expresser=mock-infiltrated tissue; silenced=Agrobacterium infiltrated and mock inoculated tissue. Two studies were carried out. Images were taken of young developing systemic leaves at various days post inoculation. Images were obtained using a confocal system (model 1024ES, BioRad) attached to an upright microscope (Zeiss Axioshop) with a 2.5× objective, as previously described by Cheng et al., 2000. DPI=days post inoculation

FIG. 9 Suppression of GFP silencing is correlated with virus accumulation. Composite image is of a stem of a plant above the leaf inoculated with M^{IC}1,3,6* and shows transient suppression of GFP silencing induced by M^{IC}1,3,6*. Values indicate virus levels in green fluorescing leaf tissue and the distal red fluorescing leaf tissue (ng virion per mg fresh weight of tissue). Values represent means and standard deviations for 3 replicates per tissue sample. Light or green areas indicate expression while dark or red areas indicate silencing of expression.

FIG. 10 Model for the mechanism of TMV spread, silencing and stabilization of unfused RNA or protein. The model indicates that host cytoplasmic silencing enzymes cannot enter viral replication complex (Virus Replication body), or the protein of the targeted RNA was made before destruction and therefore protected in the virus replication body. In either case the reporter protein is protected from destruction. Note that the virus replication body (Virus Replication Body) contains 126 kDa protein and other viral and host factors.

FIG. 11 Delay of gene silencing in transgenic N. tabacum plants expressing the 126 kDa protein:GFP fusion maps to amino acid 601 of the 126 kDa protein of TMV. Experiments were conducted using methods identical to those described to obtain results shown in FIG. 6. Images show the effect of various strains and mutants of TMV on the accumulation of GFP in the transgenic plants. When amino acid 601 was that found in the U1 sequence (i.e. as for M^{IC}m6 or U1 viruses) silencing of the transgene was delayed compared with plants inoculated with virus where amino acid 601 was that found in the M^{IC} sequence (i.e. as for U1m6 and M^{IC}). M^{IC}=progeny virus from a cDNA representing the masked strain of TMV; M^{IC}m6=progeny virus from transcript of a cDNA representing the masked strain of TMV with a single mutation at amino acid 601 resulting in a residue representing the U1 sequence; U1=virus representing the severe U1 strain of TMV; U1m6=progeny virus from transcript of a cDNA representing the U1 strain of TMV with a single mutation at amino acid 601 resulting in a residue representing the M^{IC} sequence.

FIG. 12 Effect of ectopic expression of 126 kDa protein:GFP fusion on GFP expression in epidermal cells from infiltrated leaves of N. benthamiana plants expressing GFP directed to the endoplasmic reticulum (GFPer; plant line 16c). GFP-expressing N. benthamiana leaves were infiltrated with buffer (mock), Agrobacterium tumefaciens directing expression of a GFP (GFP) with 78% identity to the transgene GFP (GFPer), a 50%/50% mixture of this Agrobacterium with one directing expression of the 126 kDa protein:GFP fusion (fusion GFP sequence was identical to that of the free GFP expressed from the binary), or with Agrobacterium directing expression of the 126 kDa protein:GFP fusion alone. In addition, plants were infiltrated with A. tumefaciens directing expression of GFPer or a 50%/50% mixture of this Agrobacterium with one directing expression of the 126 kDa protein:GFP fusion. The leaves were imaged at various days post infiltration using a confocal system and a 63× objective lens under conditions described for the previous confocal imaging work. Trt.=treatment. DPI=days post inoculation. Light or green areas indicate expression while dark areas indicate silencing of expression.

FIG. 13 Effect of ectopic expression of 126 kDa protein:GFP fusion on GFP expression in epidermal cells from infiltrated leaves of nontransgenic N. benthamiana plants. N. benthamiana leaves were infiltrated with buffer (mock), Agrobacterium tumefaciens directing expression of a GFP (GFP) with 78% identity to the transgene GFP (GFPer) described in FIG. 12, a 50%/50% mixture of this Agrobacterium with one directing expression of the 126 kDa protein:GFP fusion (fusion GFP sequence was identical to that of the free GFP expressed from the binary), or with Agrobacterium directing expression of the 126 kDa protein:GFP fusion alone. In addition, plants were infiltrated with A. tumefaciens directing expression of GFPer or a 50%/50% mixture of this Agrobacterium with one directing expression of the 126 kDa protein:GFP fusion. The leaves were imaged at various days post infiltration using a confocal system and a 63×objective lens under conditions described for the previous confocal imaging work. Trt.=treatment. DPI=days post inoculation. Light or green areas indicate expression while dark areas indicate silencing of expression.

### DETAILED DESCRIPTION OF THE INVENTION

The invention overcomes the limitations of the prior art by providing methods and compositions for modulating gene expression in plants. In particular, the inventors have identified the 126 kDa and 183 kDa proteins of a subgroup sindbis virus as being capable of modulating gene silencing of particular coding sequences, even when provided in the absence of other viral factors. The technique may find particular use for modulating expression of one or more transgenes by decreasing gene silencing, as silencing of transgenes can frequently occur, especially when transgenes are present in more than one copy in a genome. This affect may be achieved without the need for fusions between a transgene coding sequence and the 126 kDa and/or 183 kDa protein, or alternatively, using such a fusion.

The studies of the inventors identified a mutant of the masked strain of TMV, M^{IC} 1,3, altered in two amino acids within the 126/183 kDa sequence, that infects N. tabacum but produces no systemic symptoms. M^{IC}1,3 accumulated in inoculated leaves and entered the vascular tissue similarly to the parental masked (M^{IC}) strain, but failed to accumulate in systemic leaves of N. tabacum. The lack of systemic accumulation by M^{IC}1,3 in N. tabacum was due to a host RNA silencing mechanism, as determined by the presence of small (approximately 25 nucleotide) RNAs and the loss of fluorescence signal from green fluorescent protein (GFP) fused to a viral protein. Conversely, the ability of certain TMV strains and mutants to accumulate in systemic tissue was correlated with their ability to delay silencing of a viral:nonviral fused transgene, transiently suppress silencing of a non-viral transgene encoding GFP, and stabilize accumulation of 126 kDa protein in protoplasts. Therefore, the 126/183 kDa proteins suppress silencing by protecting target RNA from degradation.

It was indicated by the inventors that the 126 kDa and 183 kDa proteins could suppress silencing in the absence of other viral factors. For example, it was shown that expression of the 126 kDa protein in plants containing an unfused GFP construct exhibiting silencing in control tissues exhibited delayed silencing of GFP expression. Certain embodiments of the current invention thus concern plant transformation constructs comprising a nucleic acid sequence encoding the 126 kDa and/or 183 kDa proteins, their subgroup sindbis homologues or mutations thereof which are not provided as fusions with other coding sequences. The 126 and 183 kDa proteins, respectively, enhance or are required for virus accumulation. The 126 kDa protein contains conserved domains that by computer alignment encode methyltransferase and helicase domains surrounded by regions of unknown function and do not contain a known protease domain between them. The 183 kDa protein contains these same domains plus an RNA dependent RNA polymerase domain. All plant subgroup sindbis viruses contain these domains and by sequence comparison and position of these domains are here considered homologues of each other. Such coding sequences may be provided that are operably linked to a heterologous promoter. Expression constructs are also provided comprising these sequences, as are plants and plant cells transformed with the sequences. In further embodiments of the invention, the 126 kDa protein and/or 183 kDa protein may or may not be provided as a fusion product with a coding sequence. For example, the 126 kDa protein and/or 183 kDa protein may be fused with a coding sequence imparting a desirable phenotype to a plant.

Various examples of viral proteins that can be utilized in accordance with the present invention are included in the attached sequence listing. Applicants point out that SEQ ID NOS:8 and 9 in fact represent a single protein, connected by a single amino acid (e.g., Alanine) by virtue of a readthrough by the virus of an internal stop codon (see SEQ ID NO:7). The same situation arises with SEQ ID NOS:23 and 24 (and SEQ ID NO:22).

The construction of vectors which may be employed in conjunction with plant transformation techniques using these or other sequences according to the invention will be known to those of skill of the art in light of the present disclosure (see, for example, Sambrook et al., 1989; Gelvin et al., 1990). The techniques of the current invention are thus not limited to any particular nucleic acid sequences.

I. Transformation Vectors

Vectors used for plant transformation may include, for example, plasmids, cosmids, or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. Introduction of such sequences may be facilitated by use of bacterial or yeast artificial chromosomes (BACs or YACs, respectively), or even plant artificial chromosomes. Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al, 1987), Adh (Walker et al., 1987), sucrose synthase (Yang & Russell, 1990), a-tubulin, actin (Wang et al., 1992), cab (Sullivan et al., 1989), PEPCase (Hudspeth and Grula, 1989) or those associated with the R gene complex (Chandler et al, 1989). Tissue specific promoters such as root cell promoters (Conkling et al., 1990) and tissue specific enhancers (Fromm et al., 1986) are also contemplated to be particularly useful, as are inducible promoters such as ABA- and turgor-inducible promoters. In one embodiment of the invention, the native promoter of a coding sequence is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is contemplated that vectors for use in accordance with the present invention may be constructed to include the ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). It is proposed that the use of an enhancer element, such as the ocs element and particularly multiple copies of the element, will act to increase the level of transcription from adjacent promoters when applied in the context of plant transformation. In one embodiment of the invention, the native translation enhancer of a coding sequence is used (i.e. when expressed from within the virus genome).

It is specifically envisioned that 126 kDa protein and/or the 183 kDa protein coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, and an a-tubulin gene that also directs expression in roots.

B. Terminators

Transformation constructs prepared in accordance with the invention will typically include a 3′ end DNA sequence that acts as a signal to terminate transcription and allow for the poly-adenylation of the mRNA produced by coding sequences. In one embodiment of the invention, the native terminator of a 126 kDa protein and/or the 183 kDa protein coding sequence used (i.e. as expressed from the viral genome). Alternatively, a heterologous 3′ end may enhance the expression of the gene. Examples of terminators which are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

Many selectable marker coding regions are known and could be used with the present invention including, but not limited to, neo (Potrykus et al, 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154,204, 1985); a methotrexate resistant DHFR (Thillet et al, 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is also contemplated as a particularly useful reporter gene (Sheen et al., 1995; Haseloffet al., 1997; Reichel et al., 1996; Tian et al., 1997; WO 97/41228). Expression of green fluorescent protein may be visualized in a cell or plant as fluorescence following illumination by particular wavelengths of light.

II. Methods for Genetic Transformation

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

In certain embodiments of the invention, delivery of a nucleic acid is achieved using a Sindbis-like virus. For example, a selected coding sequence that one desires to have expressed in a cell may be introduced into the genome of a Sindbis-like virus and introduced into a plant cell via infection with the virus. In accordance with the invention, it is not necessary that the coding sequence be fused to a 126 kDa protein and/or 183 kDa protein. In this manner, a coding sequence may be expressed from a virus genome in a transient manner to allow protection for transgenic proteins in tissue where virus accumulates. This effect is well within the capabilities of the skilled artisan, as virus vectors have used to express multiple foreign genes (i.e., the protein of interest and the 126 kDa protein and or 183 kDa protein) from their genomes (Chapman et al., 1992; Hamamoto et al., 1993). Transient expression may also be utilized where the gene encoding a protein of interest in transformed into a plant cell such that the plant stably carries expresses the transgene. The virus is then used ot infect the transgenic plant tissue where virus protein accumulates and interacts with the protein of interest.

A. Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al, (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al, 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et a/, 1997), cotton (McCabe and Martinell, 1993), tomato (VanEck et al. 1995), and legumes in general (U.S. Pat. No. 5,563,055, specifically incorporated herein by reference in its entirety).

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

III. Tissue Cultures

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. Bactoagar, Hazelton agar, Gelrite, and Gelgro are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al. (1975) and MS media (Murashige and Skoog, 1962).

IV. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment: In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes. such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

It further is contemplated that the herbicide DALAPON, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. Pat. No. 5,508,468; and U.S. Pat. No. 5,508,468; each of the disclosures of which is specifically incorporated herein by reference in its entirety).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U S. Pat. No. 5,508,468.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants are preferably matured either in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

V. Breeding Plants of the Invention

In addition to direct transformation of a particular plant genotype with a construct prepared according to the current invention, transgenic plants may be made by crossing a plant having a selected DNA of the invention to a second plant lacking the construct. For example, a selected gene can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking said desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

VI. Definitions

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an Ro transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

Selected DNA: A DNA segment which one desires to introduce into a genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule capable of replication in a host cell and/or to which another DNA segment can be operatively linked so as to bring about replication of the attached segment. A plasmid is an exemplary vector.

### VII. EXAMPLES

The following examples are included to illustrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### M^{IC}1,3 Accumulation Pattern in Plant Tissue and Isolation of Mutants of M^{IC}1,3 Capable of Systemic Disease in N. tabacum

M^{IC}1,3 infects inoculated leaves of N. tabacum, but does not induce systemic symptoms on the host (N. tabacum cv. Xanthi, Shintaku et al., 1996). Compared with the parental virus, M^{IC}1,3-encoded proteins accumulated differentially in N. tabacum or in protoplasts. In inoculated leaves of N. tabacum, there was no difference in coat protein (CP) accumulation between the two viruses through the period when systemic spread of TMV normally occurs (Table 1). When M^{IC}1,3 and M^{IC} were inoculated onto N. tabacum cv. Xanthi NN, a hypersensitive host for TMV, necrotic lesion appearance and lesion diameters were identical for the two viruses. This finding indicated that the accumulation of M^{IC}1,3-encoded movement protein (MP), essential for cell-to-cell movement of this virus, was sufficiently like the parental strain to induce normal size lesions.

TABLE 1
Detection of viral coat protein in M^{IC}ml,3 or M^{IC}-TMV inoculated
Xanthi tobacco leaves at 4 and 8 DPI by ELISA

mg coat protein/g fresh leaf tissue
fresh weight leaf tissue

| DPI | M^{Ic}ml,3 | M^{IC}-TMV |
|---|---|---|
| 4 | X = 0.23 ± 0.03* | X = 0.18 ± 0.02 |
| 8 | X = 0.11 ± 0.01 | X = 0.13 ± 0.02 |

*Mean of four plants.

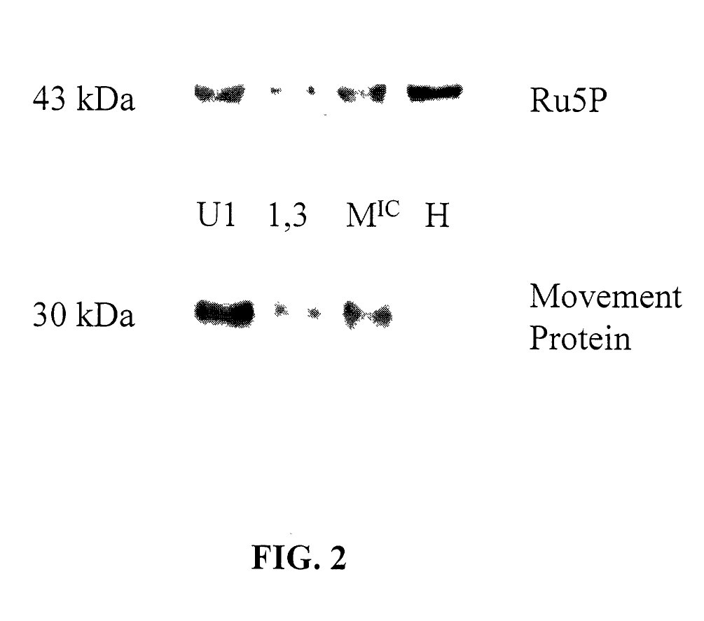

Protoplasts isolated from mature leaves and subsequently inoculated with M^{IC}1,3, M^{IC}, or U1 resulted in similar MP accumulation for all three viruses (FIG. 2). The nucleic acid sequence for the 126 kDa and 183 kDa coding sequence of the U1 strain are given in SEQ ID NO:1 and SEQ ID NO:3, and the corresponding amino acid sequences are given in SEQ ID NO:2 and SEQ ID NO:4, respectively. The accumulation of the viral 126 kDa protein, a protein containing conserved motifs for methyltransferase and helicase activities and important for modulating virus replication, decreased by 35-45% for protoplasts inoculated by M^{IC}1,3 compared to those inoculated with M^{IC} (Table 2).

TABLE 2
Relative Incorporation of ³⁵S-Label into the Viral 126-kDa Protein in
Protoplasts^{a}

| M^{IC} | M^{IC}m1,3 | M^{IC}ml,3,6 |
|---|---|---|
| 1.89^{a} | 1.0 | 2.32 |

^{a}Data were corrected to normalize differences in percentage of protoplasts infected and in protein loading per lane

Although M^{IC}1,3-inoculated protoplasts accumulated less 126 kDa protein than M^{IC}-inoculated protoplasts, the ability of M^{IC}1,3 to spread in inoculated leaves was similar to the parental M^{IC}. The appearance of M^{IC}1,3 or M^{IC} CP was monitored in minor vein cells of inoculated N. tabacum cv. Xanthi leaves during the period when systemic symptoms would normally appear. Both viruses were able to invade any of the cell types within the vascular tissue (Table 3).

TABLE 3
Percentage of infected cells for three cell types of typical
class V veins in inoculated Xanthi nn leaves

| | | | Percentage of | | |
|---|---|---|---|---|---|
| | | | cells infected | | |
| Inoculum | DPI^{a} | Number of veins* | BS^{#} | VP⁺ | C^{@} |
| Mock | 4 | 6(2) | 0 | 0 | 0 |
| M^{IC}-TMV | 4 | 18(6) | 40 | 34 | 0 |
| M^{IC}ml,3 | 4 | 8(6) | 65 | 50 | 0 |
| Mock | 8 | 2(2) | 0 | 0 | 0 |
| M^{IC}-TMV | 8 | 10(6) | 100 | 88 | 7 |
| M^{IC}ml,3 | 8 | 8(6) | 100 | 90 | 3 |

*Number in Parentheses represents No. of sections analyzed
^{#}BS #Bundle sheath cell
⁺VP = Vascular parenchyma cell
^{@}C = Companion cell
^{a}DPI = days post inoculation

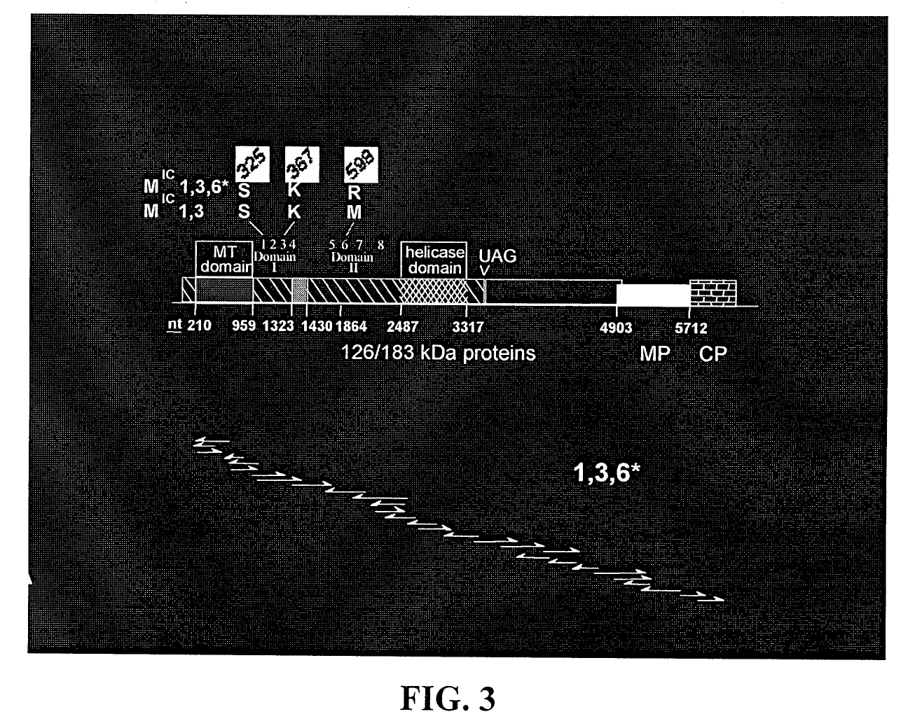

During the analysis of M^{IC}1,3 accumulation in the N. tabacum systemic host, a percentage of plants (20%, 2 of 10 plants) displayed chlorotic systemic leaves late in infection. The symptoms were much like those displayed after inoculation with M^{IC}1,3,6, a mutant virus produced in vitro by site-directed mutagenesis of the cDNA encoding M^{IC} (Shintaku et al., 1996). The systemic symptoms appeared on the same percentage of plants regardless of their possessing the U1 gene for the 183 kDa protein. Tissue was harvested from systemic leaves showing chlorotic mottling for RT-PCR™ analysis. cDNA from the systemic leaves of the transgenic plant expressing the U1 gene for the 183 kDa protein was sequenced through all the TMV orfs (FIG. 3). Virus from this tissue contained a single sequence alteration from that of the parental virus at nucleotide 1864 in the 126 kDa protein orf. The altered sequence resulted in a substitution different from the U1 sequence of arginine for methionine at amino acid residue 598 in the 126 kDa protein. cDNA from the systemic leaves of the nontransgenic plant were sequenced from nucleotides 997 to 1380, 1756-2123, and 2249-2427, an area containing all the codons resulting in amino acid differences between the M^{IC} and U1 126 kDa proteins. There was a single substitution at nucleotide 1872, altering the sequence at this position of this mutant virus to that of U1. This sequence alteration resulted in an amino acid substitution of glutamic acid for lysine at position 601 in the 126 kDa protein. Thus, both viruses, referred to as M^{IC} 1,3,1864 and M^{IC} 1,3,1872, contained single nucleotide substitutions near one another and within the 126 kDa protein and 183 kDa protein 5′ coterminal orfs.

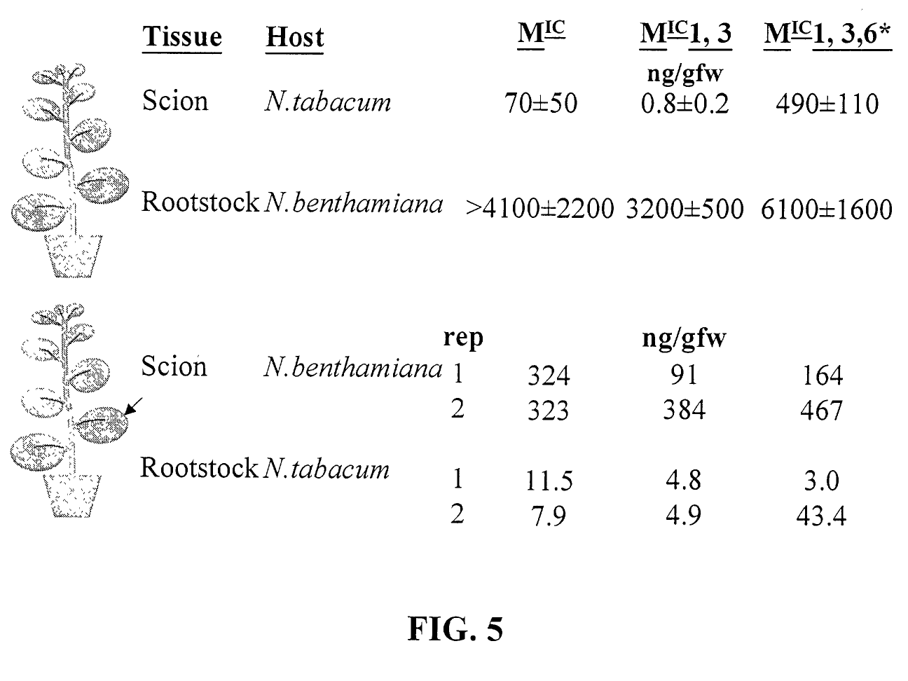

A single substitution at nucleotide 1872 of the M^{IC} cDNA U1 sequence yielded a virus, M^{IC}6, that induced a severe systemic symptom phenotype (Shintaku et al., 1996). This virus also accumulates in systemic tissue (Derrick et al., 1997). In addition, another mutant of M^{IC} was produced with altered sequences at positions 1,3 and 6 (M^{IC}m1,3,6; Shintaku et al., 1996). The symptom and systemic accumulation phenotypes induced by this virus were similar to those induced by M^{IC}1,3,1864 and M^{IC}1,3,1872. These combined results indicate that amino acids around position 601 within the TMV 126 kDa protein control systemic accumulation of TMV. The ability of M^{IC}1,3 to spread throughout the inoculated leaf and vasculature indicates that the major defect in this virus is in its ability to establish an infection in systemic leaf tissue after exit from the vasculature (as evidenced in FIG. 5). This is unlike the findings for potyviruses with an altered HC-Pro sequence. HC-Pro was determined to be essential for entry into the vascular tissue as well as its exit from this tissue (Kasschau et al., 1997). For TMV, the M^{IC} strain is attenuated or delayed in accumulation in the minor veins of the inoculated leaves compared with the U1 strain of TMV (Ding et al., 1995b). The lack of systemic virus accumulation for M^{IC}1,3 is beyond the location previously identified for the M^{IC} strain.

The positive correlation between the amount of 126 kDa protein accumulated in infected protoplasts and the ability of the viruses to accumulate systemically (Tables 2 and 3 and FIG. 5) suggested that viral activities were affected in inoculated leaves prior to entering the vascular tissue. Despite M^{IC}1,3's lower accumulation of the 126 kDa protein in inoculated protoplasts compared to the parental strain, there was no delay of spreading to and invading the vascular tissue of the inoculated leaves.

### Example 2

### MIC1,3,1864 and MIC1,3,1872 Accumulation in Inoculated Leaves and Protoplasts and, in Comparison with M^{IC}1,3, Their Accumulation in Systemic Tissue of N. tabacum

Previously, it was shown that the phenotypic equivalent of M^{IC}1,3,1872, M^{IC}m1,3,6, accumulated CP in chlorotic lesions of inoculated leaves of N. tabacum cv. Xanthi similarly to M^{IC} (Shintaku et al., 1996). CP also accumulated to similar levels in leaves inoculated with M^{IC}1,3,1864, M^{IC}, or M^{IC}1,3. Lesion diameters induced by M^{IC}1,3, 1864 and M^{IC}1,3,1872 were identical to those induced by M^{IC} and M^{IC}1,3. Therefore, adequate amounts of MP were produced to allow cell-to-cell spread of these viruses on inoculated leaves. Accumulation of 126 kDa protein in protoplasts inoculated with M^{IC} 1,3,1872, however, was similar to that observed for protoplasts inoculated with M^{IC} and more than twice that observed for protoplasts inoculated with M^{IC}1,3 (Table 2). Thus, a correlation between the ability of viruses to accumulate in systemic tissue and the level of their 126 kDa protein in infected protoplasts was observed. A similar trend was observed for other strains and mutants of TMV (Derrick et al., 1997).

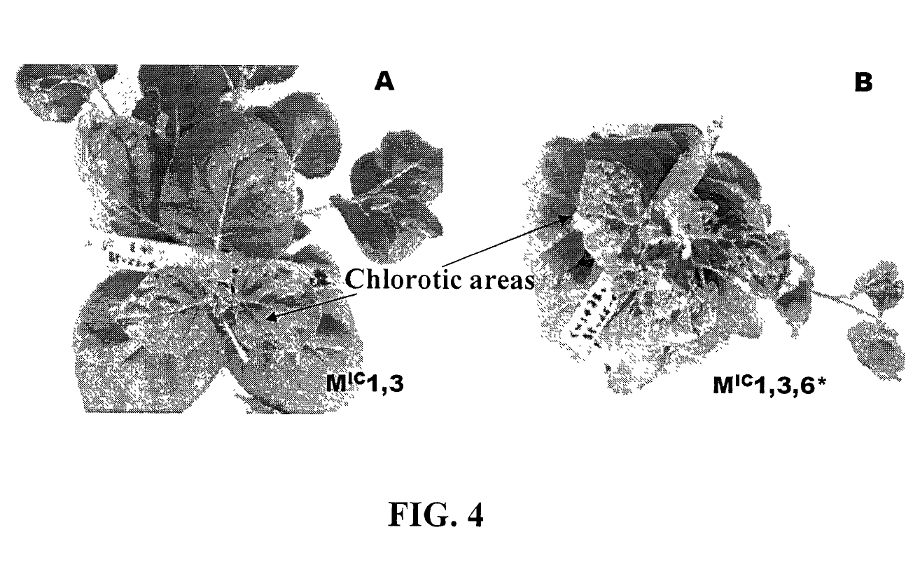

Two hosts that allowed systemic accumulation of M^{IC}1,3 were identified. Nicotiana benthamiana and Capsicum annuum plants were inoculated with virus and observed over time for systemic symptoms and virus accumulation. Sixteen days after inoculation, N. benthamiana plants inoculated with M^{IC}1,3 displayed chlorotic areas on systemic leaves (FIG. 4). These symptoms were less severe than those induced by M^{IC} or U1. C. annuum plants inoculated with M^{IC}1,3 displayed chlorotic areas on systemic leaves 6-7 days after inoculation. As was observed for the virus-infected N. benthamiana plants, the symptoms induced by M^{IC}1,3 on C. annuum were less severe than those induced by M^{IC} or U1. Systemic leaves displaying symptoms from both plant species were analyzed for the presence of virus by RT-PCR™ analysis. The nucleotide region from nucleotides 997-1380, 1756-2123, and 2249-2427 was sequenced and showed that the protein was unchanged. Extracts were also inoculated onto leaves of the systemic tobacco host and no systemic symptoms were observed through 30 days post inoculation. Therefore, the ability of M^{IC}1,3 to systemically infect N. benthamiana and C. annuum was not due to viral mutation.

Grafting studies determined that M^{IC}1,3 could enter and spread through the vascular tissue of N. tabacum. Reciprocal grafts were made between N. tabacum, the nonpermissive host for M^{IC}1,3, and N. benthamiana, the permissive host for M^{IC}1,3, and the leaves of the rootstocks were inoculated with M^{IC}1,3, M^{IC}1,3,1864 or M^{IC}. All three viruses accumulated in shoot apices of the N. benthamiana scions (FIG. 5) in a time frame similar to that observed during phloem transport (8 days post inoculation). Thus, M^{IC}1,3 can enter the sieve elements of N. tabacum and travel to shoot apices through vascular tissue. In the reciprocal graft, M^{IC}1,3 accumulated 10 fold less in the N. tabacum scion compared with M^{IC} or M^{IC}1,3,1864. A similar result was obtained by analyzing young leaves of N. tabacum (cv. Xanthi) through reverse transcription and PCR™ after inoculation of the lower part of the plant with M^{IC}1,3. M^{IC}1,3 was either not detected or detected at very low levels in systemic tissue compared with that observed after U1-TMV inoculation. Therefore, M^{IC}1,3 either had difficulty exiting vascular tissue or establishing infection after exit.

### Example 3

### Systemic Accumulation is Correlated with a Delay in Transgene Silencing

Certain viruses accumulate in inoculated leaves but fail to accumulate in systemic tissue, eventually leading to a recovered host plant (Al-Kaff et al., 1998; Covey et al., 1997; Ratcliff et al., 1997). The inventors inoculated N. benthamiana plants with M^{IC}1,3, M^{IC}, U1, and M^{IC}1,3,1864 and observed the symptom phenotype induced over time. Plants inoculated with U1 died after approximately 29 days post inoculation, but those inoculated with M^{IC}, M^{IC}1,3,1864, and M^{IC}1,3 continued growing despite symptoms ranging from severe to mild. Although plants inoculated with M^{IC} had severe symptoms in the first systemic leaves, green tissue emerged from the shoot apices that expanded normally and had little virus. Thus, the recovery phenotype displayed by N. benthamiana infected with M^{IC} demonstrated that RNA silencing may be functioning in this host against TMV.

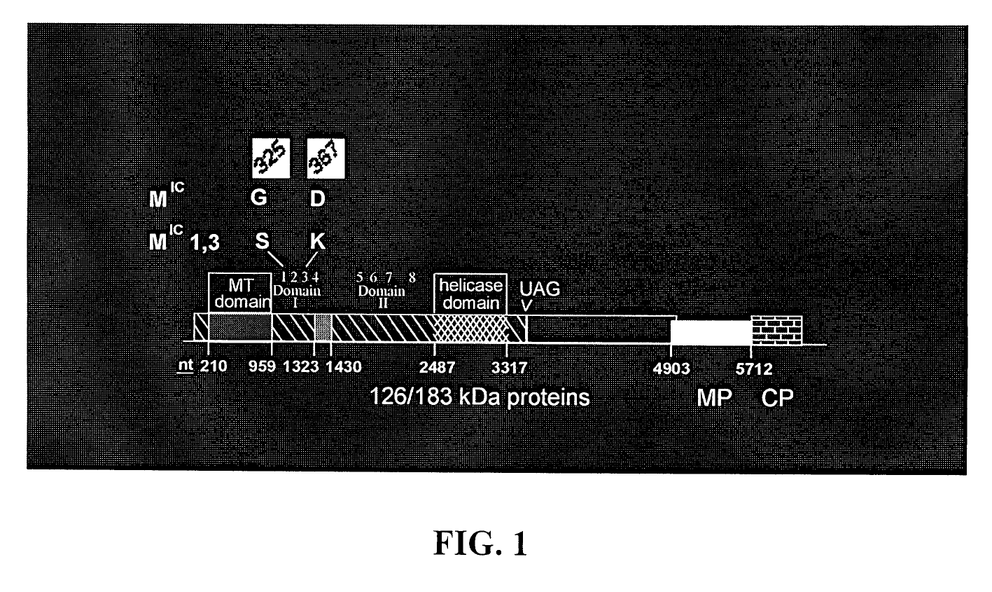

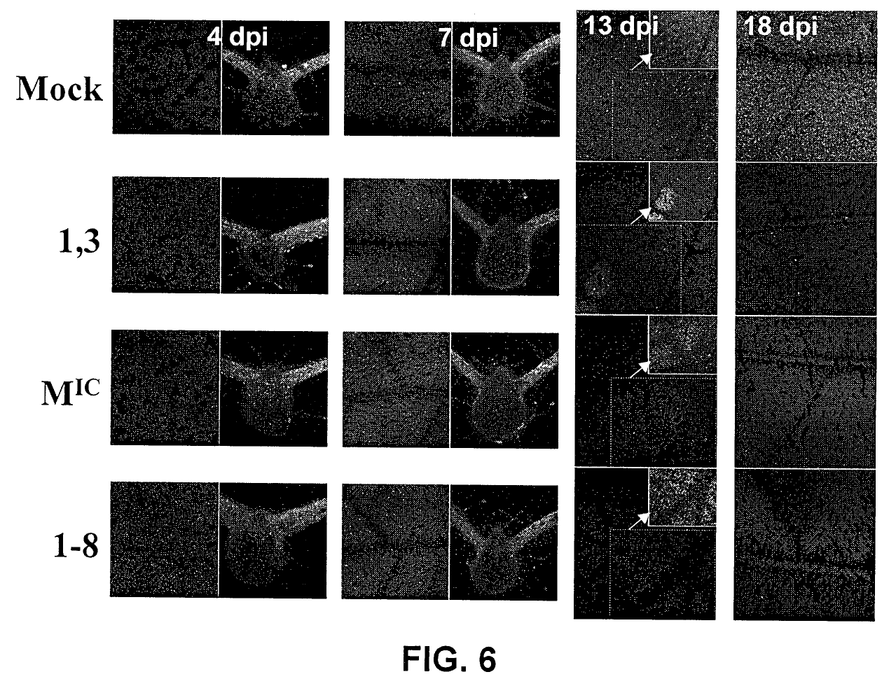

N. tabacum cv. Xanthi expressing a 126 kDa protein:green fluorescent protein (GFP) fusion were challenged with various strains and mutants of TMV and the level of GFP expression in systemic leaves monitored through confocal microscopy as the infections progressed (FIG. 6). Host-derived viral:GFP transgene expression was silenced earlier in systemic leaves of plants inoculated with M^{IC}1,3 or M^{IC} than in those inoculated with a mutant of M^{IC} where all eight amino acids differing between M^{IC} and U1 were altered to the U1 sequence, yielding M^{IC}1-8, a virus that produces U1-like severe systemic symptoms. In initial studies, it was determined that amino acid 601 (position 6 in FIG. 1) in the 126 kDa protein was a determinant for systemic accumulation of TMV (see above and Derrick et al., 1997). This was correlated with delayed silencing of transgenes. The, 126 kDa protein-GFP expressing plants inoculated with M^{IC} 6 were delayed in GFP silencing compared to M^{IC}, while a complementary mutant, UIm6, containing the amino acid from M^{IC} at position 6 in the U1 background, silenced GFP fluorescence more quickly than did U1.

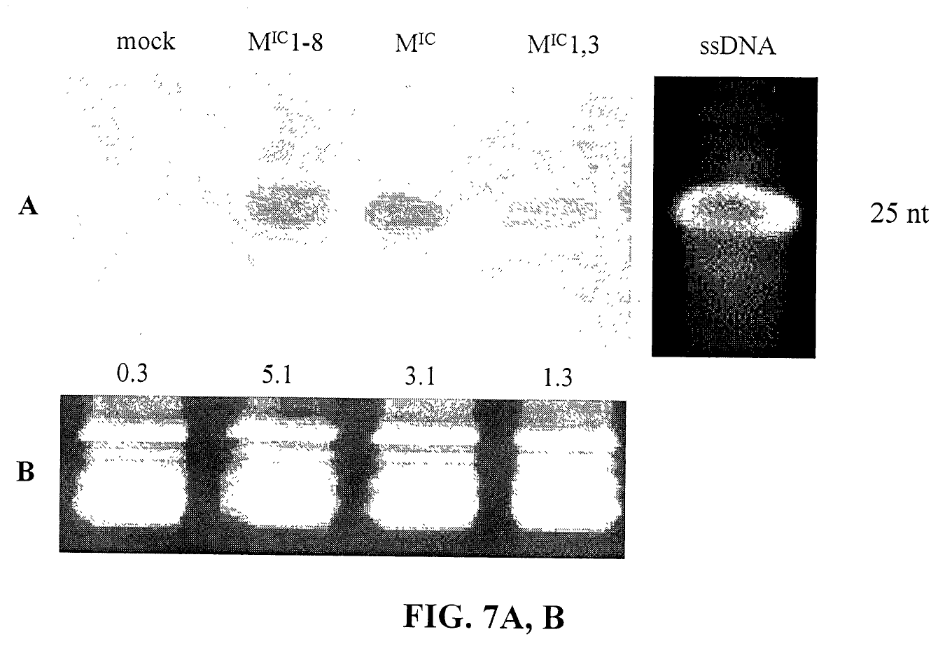

Tissue was analyzed from leaves harvested at 13 days post inoculation, when silencing was particularly apparent (FIG. 7). Tissue was harvested from leaves one above those shown in FIG. 6 and assayed for the appearance of small RNAs, indicative of active RNA silencing. Small RNAs were present in all virus challenged tissue with the quantity of small RNAs being negatively correlated with the level of GFP silencing observed (compare results in FIG. 7 with those shown in FIG. 6). These results indicated that RNA silencing was ongoing even while the protein was being protected for expression, as shown by the continued GFP expression in plants inoculated with M^{IC}1-8.

The ability of the strains and mutants of TMV to accumulate in systemic tissue was negatively correlated with the ability of the host to silence fluorescence from the 126 kDa protein:GFP fusion in transgenic N. tabacum infected with the respective viruses (Derrick et al., 1997; Shintaku et al., 1996; FIG. 6 and FIG. 7). Recently, several research groups have identified viral proteins that either suppress or prevent the silencing of transgenes (Anandalakshmi et al., 1998; Brigneti et al., 1998; Kasschau and Carrington, 1998) based on systemic virus movement (Cronin et al., 1995; Ding et al., 1995a). The 126 and/or 183 kDa proteins are grouped among these proteins on that basis (Nelson and van Bel, 1998).

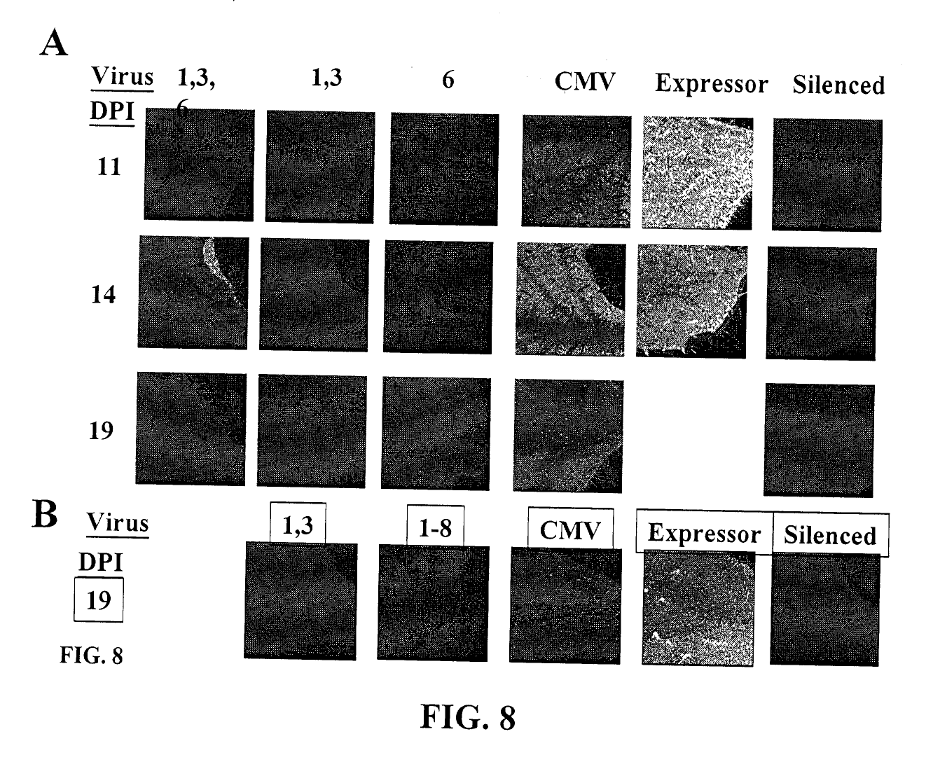

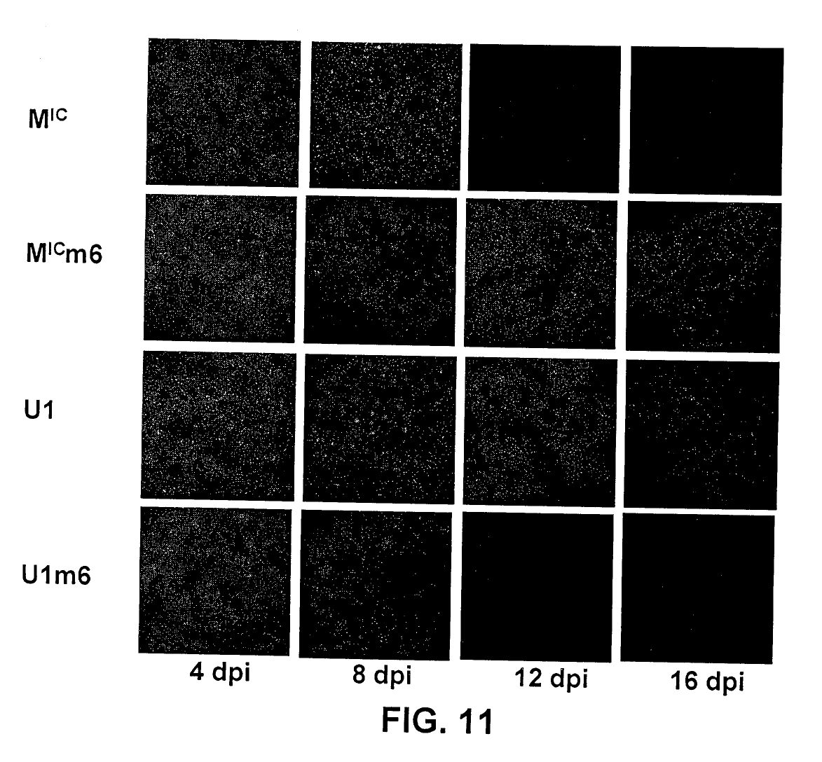

Results indicated that the 126 kDa and/or the 183 kDa protein(s) function to suppress RNA silencing or stabilize protein expression in systemic tissue. The results show that M^{IC}1,3 which efficiently silences GFP expression, can move through the vascular tissue and establish some infection in systemic tissue (FIG. 5) Thus, since all the viruses tested could move and accumulate systemically, it is not simply the presence of the virus that leads to delayed or suppressed gene silencing. Further evidence for this can be seen through the results with M^{IC}6, a virus that accumulates in systemic tissue at no greater levels than M^{IC}, but greatly delays or suppresses GFP silencing compared with M^{IC} (Derrick et al., 1997 and FIGS. 8 and 11). These observations support the hypothesis that the sequence of the 126 kDa and/or 183 kDa proteins and not simply its (their) presence in systemic tissue is responsible for the transient stabilization of GFP in the transgenic plants.

### Example 4

### Suppression of RNA Silencing of a Nonviral Transgene Maps to the 126 kDa Protein of TMV

To determine if the 126 or 183 kDa proteins of TMV could suppress RNA silencing of a nonviral transgene, seed of N. benthamiana plants expressing GFP (line 16c) were obtained from Dr. David Baulcombe (Sainsbury Laboratories, Norwich, United Kingdom). These plants can be silenced for GFP expression by infiltrating young leaves with Agrobacterium tumefaciens expressing the GFP gene from a binary vector (Ruiz et al. 1998). Plants silenced for GFP expression by Agrobacterium infiltration were later challenged in newly silenced leaves with the strains and mutants of TMV described herein. The study results indicated that M^{IC}1,3 could not suppress the silencing in systemic tissue above the inoculated leaves, while M^{IC}1-8 and other mutants containing the amino acid residue found in the U1 strain at position 6 could suppress silencing of the GFP (FIG. 8). Thus, the ability to suppress the silencing of this nonviral sequence in N. benthamiana mapped to the same protein as was found during the delay in silencing of the viral:nonviral transgene fusion expressed in N. tabacum (compare results in FIG. 6 with those in FIG. 8).

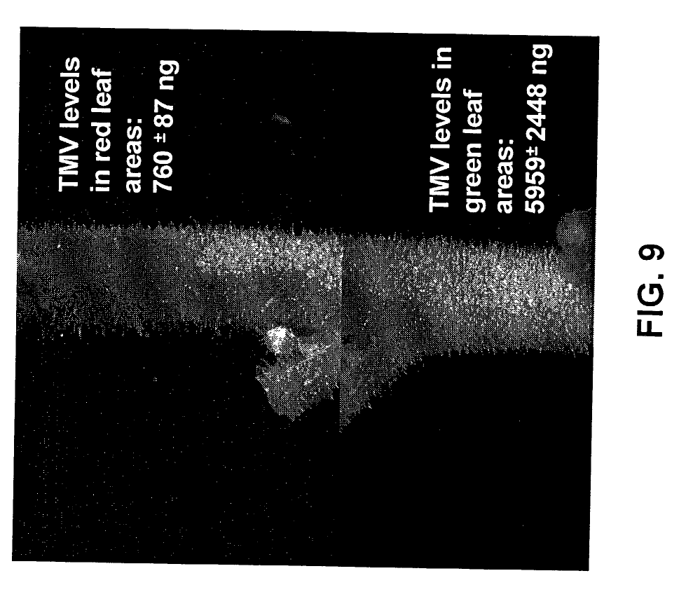

Newly developing leaf tissue was observed over time in the challenged 16c plants. Virus that produced severe symptoms in N. benthamiana prevented continued leaf expansion and plant growth, making it difficult to determine the stability of the suppression in these plants. However, M^{IC}1,3,6, which produces mild symptoms, allowed continued plant growth and transient suppression of silencing (FIG. 9).

### Example 5

### A Model for TMV Silencing and Suppression

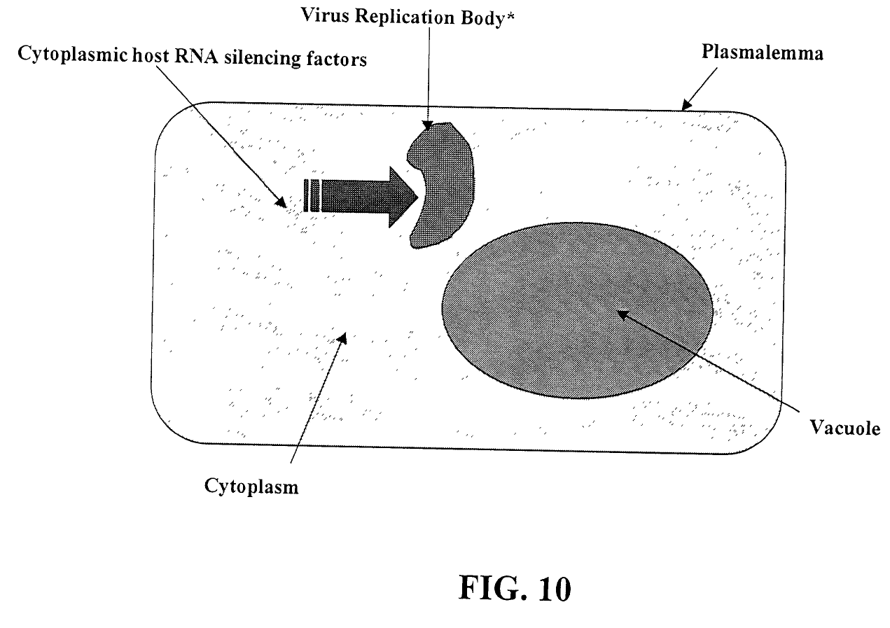

From the results of this study, it is possible to propose a model explaining the accumulation patterns of the TMV strains and mutants (FIG. 10). The most attenuated virus in systemic accumulation, M^{IC}1,3, entered the vascular tissue, spread to the shoot apex and exited this tissue. At this point, the virus, must establish an infection Interestingly, M^{IC}1,3, the virus that induced the most rapid silencing phenotype, yielded the lowest level of small RNAs while viruses that delayed silencing induced the highest levels of small RNAs. Thus each TMV strain, regardless of its ability to accumulate to high levels in systemic tissue, could induce the RNA silencing pathway in systemic tissue. The presence of small RNAs in systemic tissue of plants infected with viruses that induce severe symptoms suggests that these viruses accumulate despite active silencing of targeted host and viral sequences. In the case of U1, M^{IC}6 and the other viruses that establish infection in the systemic tissue, the 126 kDa protein functions to stabilize its own RNA and protein (Table 2 and Derrick et al., 1997) as well as homologous transgene messages and/or their proteins. The homologous protein to the 126 kDa protein encoded by Brome mosaic virus, 1a, stabilizes RNA accumulation (Sullivan and Ahlquist, 1999). Stabilization of RNA and/or protein expression by these viral proteins in turn may allow infection to progress and symptoms to develop. Thus, the ability of TMV to accumulate depends on its ability to avoid the host proteins involved in silencing, rather than to disable the silencing system. Additional support for this model comes from the observation that the suppression of GFP silencing was transient, being dependent on the active accumulation of virus. This active virus accumulation necessarily includes the accumulation of the 126 and/or 183 kDa proteins which, in the model, act to form secluded areas that trap proteins and protect them from degradation. It is known that TMV produces cytoplasmic bodies associated with virus accumulation that contain large amounts of 126 kDa protein (e.g. Szecsi et al. 1999). These bodies could trap viral and nonviral RNA and protect it from degrading proteins involved in RNA silencing.

### Example 6

### The 126 kDa Protein Alone can Suppress Silencing of GFP

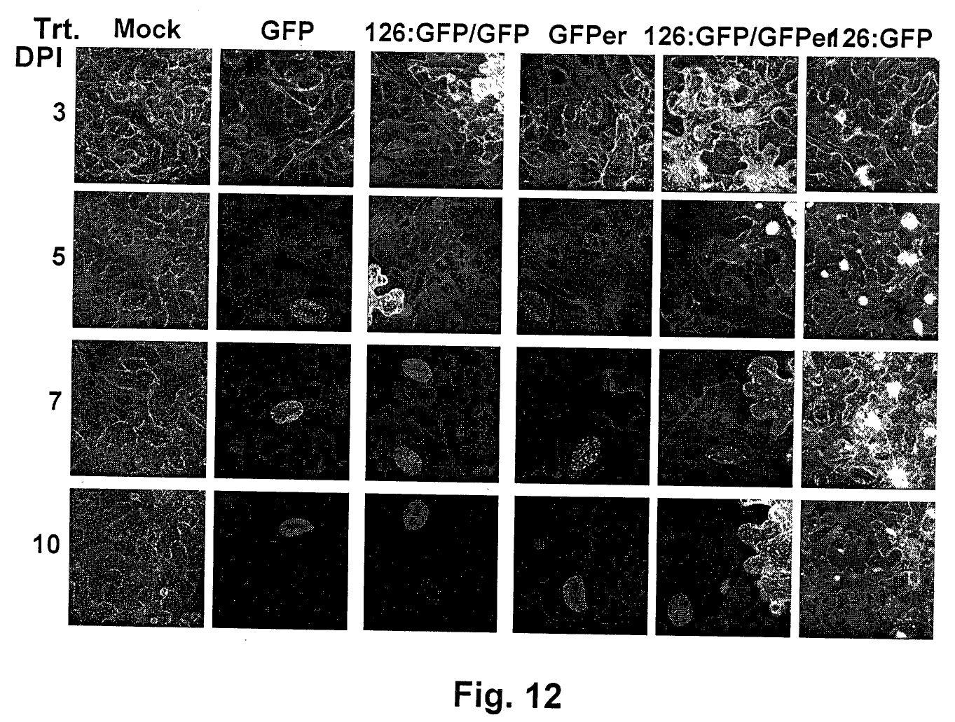

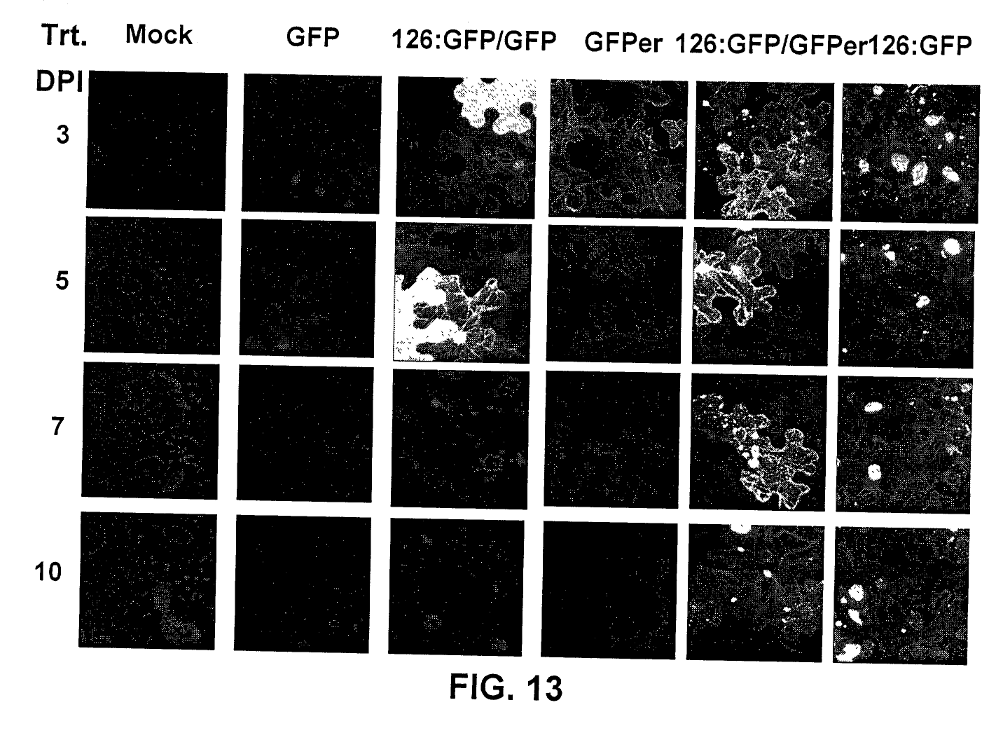

To determine whether the 126 kDa protein could suppress silencing in the absence of other viral factors, constructs of the 126 kDa protein fused with GFP were agroinfiltrated into leaves of N. benthamiana 16c plants expressing GFP or N. benthamiana plants that were not transformed. Because the GFP expressed by the 126 kDa protein:GFP fusion (referred to hereafter as GFP) was not identical in sequence or subcellular location to the GFP expressed in the 16c plants (referred to hereafter as GFPer; 78% sequence identity between these GFPs) nontransgenic N. benthamiana was infiltrated and transformed with GFP that was not fused to the 126 kDa protein to determine its ability to silence itself and the GFPer transcript in the transgenic plants. Tissue infiltrated with Agrobacterium containing the unfused GFP construct silenced both itself and the transgene by 5 days post infiltration, whereas tissue infiltrated with 126 kDa protein:GFP and unfused GFP delayed silencing of GFP expression in both nontransformed and transformed plants (FIGS. 12, 13; images in 126:GFP/GFP column compared with those in GFP column). A similar delay in silencing also occurred in both transgenic and nontransgenic plants infiltrated with 126 kDa protein:GFPer compared with GFPer alone (FIGS. 12, 13; images in 126:GFP/GFPer column compared with those in GFPer column). This indicated that the 126 kDa protein, in the absence of other viral factors, delayed the loss of GFP expression either by inhibiting protein or RNA degradation.

### Example 7

### Materials and Methods

Virus Strains and Mutants

The “masked” (M) and U1 strains of TMV were obtained from previously described sources (Holt et al., 1990). M^{IC}-TMV refers to the progeny of infectious transcript produced from a cDNA clone of the M strain (Holt et al., 1990). M^{IC}1,3, M^{IC}1-8, M^{IC}m6, U1m6 and M^{IC}1,3,6 were produced as described (Shintaku et al., 1996). Throughout the text, M^{IC}m1,3=M^{IC} 1,3 and for all other mutants M^{IC}mX=M^{IC}X.

Plants

Nicotiana tabacum cv. Xanthi, N. tabacum cv. Xanthi NN (hypersensitive host), N. benthamiana and Capsicum annuum L. cv Marengo were used. N. benthamiana line 16c transformed to express GFP from behind a 35S promoter (Brigneti et al., 1998). N. tabacum cv. Xanthi transformed to express a fusion of the 126 kDa protein with the enhanced green fluorescent protein (GFP) from behind an enhanced 35S promoter is described below.

Antibodies

Antibodies against the movement protein (MP) and the coat protein (CP) were provided or produced as described (Derrick et al., 1997). Antiserum against ribulose-5-phospahet kinase (Ru5P kinase) was from USDA-ARS Western Cotton Research Lab, Phoenix, Ariz.

Growth of Nicotiana tabacum and Nicotiana benthamiana and Inoculation with Virus

N. tabacum cv. Xanthi or Xanthi NN, C. annuum and N. benthamiana were germinated and grown as described for N. tabacum (Ding et al., 1995b), and cuttings of N. tabacum cv. Xanthi transformed to express the 126 kDa protein:GFP fusion were grown (Ding et al., 1995b). In vitro transcripts of virus cDNAs were produced and inoculated according to Shinataku et al. (1996). After virus inoculation, plants were either left in a greenhouse under previously described conditions (Nelson et al., 1993) or placed in a growth chamber (Ding et al., 1995b). Virus was inoculated as described (Nelson et al., 1993). In vitro transcripts were produced and inoculated as described (Shintaku et al., 1996).

Necrotic Lesion Measurements

Necrotic lesion diameters were measured with a micrometer using a previously described experimental design (Bao et al., 1996).

Immunoblots and ELISA

For immunoblots shown in FIG. 2, protoplasts were harvested, extracted and analyzed as described (Derrick et al., 1997) Blots were first probed with antibody against the MP and then stripped and probed with antibody against Ru5P kinase

For ELISA analysis, tissue was harvested at the particular developmental stage and dpi as described above and the fresh weight was recorded. Tissue was extracted and ELISA conducted for CP accumulation as described for virus accumulation in transgenic tobacco expressing MP (Derrick et al., 1997).

Immunocytochemistry

To visualize virus accumulation in vascular cells from inoculated leaves (Table 3), leaf tissue was randomly sampled from virus- and mock-innoculated leaves. Tissue from N. tabacum was analyzed by double-sided labeling immunocytochemistry and light microscopy as described (Ding et al., 1996 and 1996b).

Protoplast Isolation, Inoculation and Analysis

N. tabacum cv. Xanthi used as a source for leaf-derived protoplasts were grown and maintained as described (Derrick et al., 1997). Protoplasts enriched in palisade mesophyll cells were prepared essentially as described by Kubo and Takanami (1979). Virus inoculation of protoplasts was conducted as described (Derrick et al., 1997). Immunoblot detection of MP accumulation in protoplasts and pulse-labeling of viral proteins were performed as described (Derrick et al., 1997). For protoplasts subjected to pulse radiolabelling, the incubation medium contained 80 μg of actinomycin D per ml and radiolabelling occurred from 8-10 hr. post-inoculation (Derrick et al., 1997).

Isolation, Purification and Sequencing of M^{IC} 1,3 Mutants

Progeny virus was sequenced after isolation of total RNA from systemically-infected leaves as described (Shintaku et al., 1996).

Grafting Studies

N. tabacum cv. Xanthi and N. benthamiana plants were grown as described (Ding et al. 1995b). Reciprocal grafts were made between species using a wedge grafting system as described (Kasshau et al. 1997). The grafted rootstock and scion were covered with a clear plastic lid to reduce transpiration demand on the recovering plants. After recovery and removal of the plastic lid all leaves on the scion, except those less than 2 cm in midrib length, were removed to remove extraneous sink tissue. The first and second leaves down from the graft union on the rootstock were challenged with virus one or two days after removal of the scion leaves. Virus infectivities were equalized previous to inoculation of the grafted plants by bioassay with a hypersensitive host (N. tabacum cv. Xanthi NN).

Production of Transgenic Plants Expressing the 126 kDa Protein: GFP Fusion

The cDNA fragment encoding the 126 kDa protein of TMV was produced using the “WFP” construct from M^{IC}m2 described in international patent application PCT/USO1/22390, the disclosure of which is specifically incorporated herein by reference in the entirety. The fusion protein construct was moved into the intermediate plasmid, pRTL2, as described in the patent application. The construct used to transform plants, referred to as the WFP construct, was then spliced from pRTL2 by digestion with restriction enzymes and ligated into vector pGA482 at the HindIII sites. Agrobacterium tumefaciens (LBA 4404) was then transformed with the binary vector using a modification of the method described by An et al. (1988). This modification includes after freezing in liquid nitrogen and then thawing at 37° C. for 5 min, 1 ml of YEP medium added to the tube and the cells incubated for 1 h at 28° C. Kanamycin (1 μl/ml) and nfampicin (1 μg/ml) were added and the cells incubated at 28° C. for another 2 h. The cells were centrifuged and resuspended in 50 μl of YEP medium containing the antibiotics as described above. The cells were inoculated onto YEP agar plate containing 50 μg/ml kanamycin and 10 μ/ml rifampicin, and incubated at 28° C. for 2-3 days. Leaf discs from N. tabacum cv. Xanthi were transformed using Agrobacterium tumefaciens containing the 126 kDa protein:GFP fusion through standard protocols (Horsch et al. 1988). A line putatively containing the 126 kDa protein:GFP fusion (line 1-1) was screened for the presence of the insert and expression of the transgene at the RNA and protein level (see FIG. 6 for expression pattern of GFP under the confocal microscope). The line contained multiple inserts and thus was used as cuttings for all challenge studies with virus. The cuttings were developmentally matched by their plastochron indices for all studies to reduce plant to plant variability between treatments (Nelson et al. 1993).

Agrobacterium Infiltration

A GFP sequence (eGFP, Clontech, Palo Alto, Calif., USA) cloned between an enhanced 35S promoter and 35S terminator in the binary plasmid, pRTL2 (Carrington and Freed, 1990), (construct described in Itaya et al. 1997) and the 126 kDa protein:GFP sequence used to transform N. tabacum cv. Xanthi (see above) were used for Agrobacterium infiltration studies. The binary vector containing the GFP construct was transformed into Agrobacterium tumefaciens strain LBA 4404 as described above for pRTL2 containing the 126 kDa protein:GFP fusion. LBA4404 containing either binary vector was grown under selection to an OD of 0.5, allowed to sit at room temperature for 2-3 hours without shaking and then infiltrated independently or equally mixed into the adaxial side of mature leaves of N. benthamiana line 16c as described (Voinnet et al. 1998, English et al. 1997).

Confocal Microscopy on Plants Expressing the 126 kDa Protein: GFP Expressing Fusion or the Free GFP After Challenge with Tobacco Mosaic Virus

GFP expression after virus challenge was monitored using a confocal microscope under described settings (Cheng et al. 2000).

Epifluorescence Microscopy

GFP expression from stem tissue (FIG. 9) was monitored using an epifluorescence SZX12 stereomicroscope (Olympus, Mehlville, N.Y.) attached to a spot RT digital camera (Diagnostic Instruments, Sterling Heights, Mich.). Images were collected on a PC (Dell).

Small RNA Detection

Small RNAs were detected as described in Itaya et al. (2001).

### REFERENCES

The following references, to the extent that they provide exemplary procedural or other details supplementary to those set forth herein, are specifically incorporated herein by reference.

U.S. Pat. No. 4,535,060

U.S. Pat. No. 5,302,523

U.S. Pat. No. 5,322,783

U.S. Pat. No. 5,384,253

U.S. Pat. No. 5,464,765

U.S. Pat. No. 5,508,184

U.S. Pat. No. 5,508,468

U.S. Pat. No. 5,538,877

U.S. Pat. No. 5,538,880

U.S. Pat. No. 5,545,818

U.S. Pat. No. 5,550,318

U.S. Pat. No. 5,563,055

U.S. Pat. No. 5,591,616

U.S. Pat. No. 5,610,042

EPA App. 154,204, 1985

PCT App. WO 97/41228

PCT App. WO 94/09699

PCT App. WO 95/06128

PCT App. WO 97/04103

PCT App. WO 92/17598

Abdullah et al., Biotechnology, 4:1087, 1986.

Al-Kaff et al., Science, 279:2113-2115, 1998.

An et al., In: Plant Molecular Biology Manual, Gelvin et al., (eds.), Kluwer Academic Publishers, Dordrecht, The Netherlands, 1988.

Anandalakshmi et al., Proc. Natl. Acad. Sci. USA, 95:13079-13084, 1998.

Bao et al., J. Virology, 70:6378-6383, 1996.

Bates, Mol. Biotechnol., 2(2):135-145, 1994.

Battraw and Hall, Theor. App. Genet., 82(2):161-168, 1991.

Baulcombe, Cum. Opin. Plant Biol., 2:109-113, 1999.

Bevan et al., Nucleic Acids Research, 11(2):369-385, 1983.

Bhattacharjee; An; Gupta, J. Plant Bioch. and Biotech., 6:(2):69-73. 1997.

Bonneau et al., J. Virology, 244:79-86, 1998.

Bower et al., Plant J., 2:409-416. 1992.

Brigneti et al., EMBO J., 17:6739-6746,1998.

Buchanan-Wollaston et al., Plant Cell. Repts., 11:627-631. 1992

Buising and Benbow, Mol. Gen. Genet., 243(1):71-81. 1994.

Callis, Fromm, Walbot, Genes Dev., 1:1183-1200, 1987.

Carrington and Freed, J. Virology, 64,1590-1597, 1990

Carrington et al., J. Virology, 281:1-5, 2001.

Casa et al., Proc. Natl. Acad. Sci. USA, 90(23):11212-11216, 1993.

Chandler et al., Plant Cell., 1: 1175-1183, 1989.

Chapman et al., Plant Journal 2:549-557, 1992.

Cheng et al., Plant J. 23:349-362, 2000.

Christou; et al., Proc. Natl. Acad. Sci. USA, 84(12):3962-3966, 1987.

Chu et al., Scientia Sinica, 18:659-668, 1975.

Conkling et al., Plant Physiol., 93:1203-1211, 1990.

Covey et al., Nature, 385:781-782, 1997.

Cronin et al., Plant Cell., 7:549-559, 1995.

De Block et al., EMBO J., 6(9):2513-2518, 1987.

De Block et al., Plant Physiol., 91:694-701, 1989.

De Jong and Ahlquist, J. Virology, 69:1485-1492, 1995.

Dellaporta et al., In: Chromosome structure and function: Impact of New Concepts, 18th Stadler Genetics Symposium, 11:263-282, 1988.

Deom et al., Protoplasma, 199:1-8, 1997.

Derrick et al., Mol. Plant Microbe Interact., 10:589-596, 1997.

D'Halluin et al., Plant Cell., 4(12):1495-1505, 1992.

Ding et al., BioTechniques, 20:111-115, 1996b.

Ding et al., EMBO J., 14:5762-5772, 1995a.

Ding et al., Mol. Plant Microbe Interact., 8:32-40, 1995b.

Ding et al., Proc. Natl. Acad. Sci. USA, 93:11155-11160, 1996.

Ding et al., Proc. Natl. Acad. Sci. USA, 93:7470-7474, 1996a.

Ebert et al., 84:5745-5749, Proc. Nat'l Acad. Sci. USA, 1987.

Ellis et al., EMBO J., 6(11):3203-3208, 1987.

English et al., Plant J., 12, 597-603, 1997.

Fraley et al., Bio/Technology, 3:629-635, 1985

Fromm et al., Nature, 319:791-793, 1986.

Galli et al., Plant Cell., 1:301-311, 1989.

Gelvin et al., In: Plant Molecular Biology Manual, 1990.

Ghosh-Biswas et al., J. Biotechnol., 32(1):1-10, 1994.

Hagio et al., Plant Cell Rep., 10(5):260-264, 1991.

Hamilton and Baulcombe, Science, 286:950-952, 1999.

Hamamoto et al., Biotechnology 11 :930-932, 1993

Haseloff et al., Proc. Natl. Acad. Sci. USA, 94(6):2122-2127, 1997.

He et al., Plant Cell. Reports, 14 (2-3): 192-196, 1994.

Hensgens et al., Plant Mol. Biol., 22(6):1101-1127, 1993.

Hiei et al., Plant. Mol. Biol., 35(1-2):205-218, 1997.

Hinchee et al., Bio/technol., 6:915-922, 1988.

Holmes, Phytopathology, 24:845-873,1934.

Holt et al., Mol. Plant Microbe Interact., 3:417-423, 1990.

Hong et al., J. Virology, 228:383-387, 1997.

Horsch et al., In: Plant Molecular Biology Manual. Kluwer Academic Publishers. Dordrecht, The Netherlands, 1988.

Hou and Lin, Plant Physiology, 111: 166, 1996.

Hudspeth and Grula, Plant Mol. Biol., 12:579-589, 1989.

Ikuta et al., Bio/technol., 8:241-242, 1990.

Ishidia et al., Nat. Biotechnol., 14(6):745-750, 1996.

Itaya et al., Mol. Plant Microbe Interact., 14(11):1332-1334, 2001.

Itaya et al., Plant J., 12, 1223-1230, 1997.

Ji and Ding, Mol. Plant-Microbe Interact., 14:715-724, 2001.

Kaeppler et al., Plant Cell. Repts., 9: 415-418, 1990.

Kaeppler et al., Theor. Appl. Genet., 84(5-6):560-566, 1992.

Kasschau and Carrington, Cell, 95:461-470, 1998.

Kasschau et al., J. Virology, 228:251-262, 1997.

Katz et al., J. Gen. Microbiol., 129:2703-2714, 1983.

Klee, Yanofsky, Nester, Bio/Technology, 3(7):637-642, 1985.

Knittel et al., Plant Cell. Repts., 14(2-3):81-86, 1994.

Kubo and Takanami, J. Gen. Virol., 42:387-398, 1979.

Lakshman and Gonsalves, Phytopathology, 75:758-762, 1985.

Lawton et al., Plant Mol. Biol., 9:315-324, 1987.

Lazzeri, Methods Mol. Biol., 49.95-106, 1995.

Lee et al., Korean J. Genet., 11(2):65-72, 1989.

Lindbo et al., Plant Cell., 5:1749-1759, 1993.

Llave et al., Proc. Natl. Acad. Sci. USA, 97:13401-13406, 2000.

Lorz et al., Mol. Gen. Genet., 199:178-182, 1985.

Lucy et al., EMBO J., 19.1672-1680, 2000.

Mallory et al., Plant Cell., 13:571-583, 2001.

Marcotte et al., Nature, 335:454, 1988.

Matthews, In: Plant Virology, San Diego: Academic Press, 1991.

McCabe and Martinell, Bio/Technology, 11(5):596-598, 1993.

McCormac et al., Euphytica, 99 (1) 17-25, 1998.

Murakami et al., Mol. Gen. Genet, 205:42-50, 1986.

Nagatani et al., Bio/Technology, 11(7):471-473, 1997.

Nelson and van Bel, Prog. Bot., 59:476-533, 1998.

Nelson et al., Mol. Plant Microbe Interact., 6:45-54, 1993.

Odell et al., Nature, 313:810-812, 1985.

Ogawa et al., Sci. Rep., 13:42-48, 1973.

Omirulleh et al., Plant Mol. Biol., 21(3):415-428, 1993.

Ow et al., Science, 234:856-859, 1986.

Palauqui and Elmayan, EMBO J., 16:4738-4745, 1997.

Potrykus et al., Mol. Gen. Genet., 199:183-188, 1985.

Prasher et al., Biochem. Biophys. Res. Commun., 126(3):1259-1268, 1985.

Ratcliff et al., Science, 276:1558-1560, 1997.

Reichel et al., Proc. Natl. Acad. Sci. USA, 93 (12) p. 5888-5893. 1996.

Revers et al., Mol. Plant Microbe Interact., 12:367-376, 1999.

Rhodes et al., Methods Mol. Biol., 55:121-131, 1995.

Ritala et al., Plant Mol. Biol., 24(2):317-325, 1994.

Rogers et al., Methods Enzymol., 153:253-277, 1987.

Roossinck and Palukaitis, Mol. Plant Microbe Interact., 3: 188-192, 1990.

Ruiz et al., Plant Cell., 10:937-946, 1998.

Sambrook et al., In:: Molecular Cloning-A Laboratory Manual (second edition), Cold Spring Harbour Laboratory Press, 1989.

Scholthof et al., J. Virology, 213:425-438, 1995

Sheen et al., Plant J., 8(5):777-784, 1995.

Shintaku et al., J. Virology, 221:218-225, 1996.

Singsit e al., Transgenic Res., 6(2):169-176, 1997.

Smith et al., Plant Cell., 6:1441-1453, 1994.

Spencer et al., Plant Molec. Biology, 18:201-210, 1992.

Stalker et al., Science, 242:419-422, 1988.

Sullivan and Ahlquist, J. Virology, 73:2622-2632, 1999.

Sullivan et al., Mol. Gen. Genet., 215(3):431-440, 1989.

Sutcliffe, Proc. Natl. Acad. Sci. USA, 75:3737-3741, 1978.

Szecsi et al., Mol. Plant Microbe Interact., 12:143-152, 1999.

Thillet et al., J. Biol. Chem., 263:12500-12508, 1988.

Thomas et al., Plant Sci., 69:189-198, 1990.

Thompson et al., Euphytica, 85(1-3):75-80, 1995.

Thompson et al., EMBO J., 6(9):2519-2523, 1987.

Tian, Sequin, Charest, Plant Cell. Rep., 16:267-271, 1997.

Tingay et al., Plant J., 11(6):1369-1376. 1997.

Tomes et al., Plant Mol. Biol. 14(2):261-268, 1990.

Torbet et al., Crop Science, 38(1):226-231, 1998.

Torbet et al., Plant Cell. Repts., 14(10):635-640, 1995.

Toriyama et al., Theor. Appl. Genet., 73:16, 1986.

Traynor et al., J. Virology, 65:2807-2815, 1991.

Tsukada et al., Plant Cell Physiol., 30(4)599-604, 1989.

Twell et al., Plant Physiol., 91:1270-1274, 1989.

Uchimiya et al., Mol. Gen. Genet., 204:204, 1986.

Van Eck et al., Plant Cell. Repts., 14(5):299-304, 1995.

Vance and Vaucheret, Science, 292:2277-2280, 2001.

Vasil et al., Plant Physiol., 91: 1575-1579, 1989.

Voinnet et al., Cell, 103:157-167, 2000.

Voinnet et al., Cell, 95: 177-187, 1998.

Voinnet et al., Proc. Natl. Acad. Sci. USA, 96.14147-14152, 1999.

Walker et al., Proc. Natl. Acad. Sci. USA, 84:6624-6628, 1987.

Wang et al., Molec. Cell. Biol., 12(8) 3399-3406, 1992.

Weiland and Edwards, J. Virology, 201:116-126, 1994.

Yamada et al., Plant Cell. Repts., 4.85, 1986

Yang and Russell, Proc. Natl. Acad. Sci. USA, 87:4144-4148, 1990.

Zamore et al., Cell, 101:25-33, 2000.

Zheng and Edwards, J. Gen. Virol., 71:1865-1868, 1990.

Zhou et al., Plant Cell, Repts., 12(11).612-616, 1993.

Zukowsky et al., Proc. Natl. Acad. Sci. USA, 80:1101-1105, 1983.

What is claimed is:

1. A method of suppressing gene silencing and/or stabilizing expression of a coding sequence in a cell comprising expressing a 126 kDa protein and/or the 183 kDa protein of a subgroup sindbis plant virus in said cell.

2. The method of claim 1, comprising expressing said 126 kDa protein in said cell.

3. The method of claim 1, comprising expressing said 183 kDa protein in said cell.

4. The method of claim 1, wherein expressing comprises infecting said cell with a subgroup sindbis plant virus encoding said 126 kDa protein and/or the 183 kDa protein or homologue and allowing said 126 kDa protein and/or the 183 kDa protein or homologue to be expressed.

5. The method of claim 1, wherein said coding sequence was introduced into the genome of said cell or a progenitor thereof by genetic transformation.

6. The method of claim 5, wherein said coding sequence is present in more than one copy in said cell.

7. The method of claim 1, wherein expressing comprises transforming said cell or a progenitor thereof with a nucleic acid sequence encoding said 126 kDa protein and/or said 183 kDa protein.

8. The method of claim 1, wherein the cell is a plant cell.

9. The method of claim 8, wherein the plant cell is comprised in a plant.

10. The method of claim 9, wherein the plant is a dicotyledonous plant.

11. The method of claim 10, wherein the dicotyledonous plant is selected from the group consisting of Nicotiana spp., tomato, potato, soybean, cotton, canola, alfalfa, sunflower, and cotton.

12. The method of claim 11, wherein said plant is selected from the group consisting of Nicotiana tabacum and Nicotiana benthamiana.

13. The method of claim 9, wherein the plant is a monocotyledonous plant.

14. The method of claim 13, wherein the monocotyledonous plant is selected from the group consisting of wheat, maize, rye, rice, oat, barley, turfgrass, sorghum, millet and sugarcane.

15. The method of claim 4, wherein said subgroup sindbis plant virus is selected from the group consisting of Tobamoviruses, Tobraviruses, Hordeiviruses, Bromoviridae, Benyviruses, Idaeoviruses, Potexviruses, Allexiviruses, Foveaviruses, Pomoviruses, Carlaviruses or Vitiviruses.

16. The method of claim 1, wherein said coding sequence is expressed from the plant's genome.

17. The method of claim 4, wherein said virus comprises said coding sequence.

18. The method of claim 1, wherein said nucleic acid sequence encoding said 126 kDa protein and/or said 183 kDa protein is not fused to said coding sequence.

19. A method of delivering a polypeptide of interest to a limited part of a plant comprising the step of infecting a plant with a subgroup sindbis plant virus, wherein said virus encodes a homologue of the 126 kDa and/or 183 kDa protein and wherein said virus encodes said polypeptide.

20. The method of claim 19, wherein the virus encodes the 126 kDa protein.

21. The method of claim 19, wherein the virus encodes the 183 kDa protein.

22. The method of claim 19, wherein one or more mutations are at position 598 or 601 of M^{IC} sequence.

24. The method of claim 23, wherein the amino acid at position 598 is arginine and amino acid at position 601 is lysine.

26. The method of claim 25, wherein the amino acid at position 601 is glutamic acid and amino acid at position 598 is methionine.

27. The method of claim 19, wherein the plant is a dicotyledonous plant.

28. The method of claim 27, wherein the dicotyledonous plant is selected from the group consisting of Nicotiana spp., tomato, potato, soybean, cotton, canola, alfalfa, sunflower, and cotton.

29. The method of claim 28, wherein said plant is selected from the group consisting of Nicotiana tabacum and Nicotiana benthamiana.

30. The method of claim 19, wherein the plant is a monocotyledonous plant.

31. The method of claim 30, wherein the monocotyledonous plant is selected from the group consisting of wheat, maize, rye, rice, oat, barley, turfgrass, sorghum, millet and sugarcane.

33. The method of claim 27, wherein said plant is selected from the group consisting of Nicotiana tabacum and Nicotiana benthamiana.

34. The method of claim 19, wherein said subgroup sindbis plant virus is selected from the group consisting of Tobamoviruses, Tobraviruses, Hordeiviruses, Bromoviridae, Benyviruses, Idaeoviruses, Potexviruses, Allexiviruses, Foveaviruses, Pomoviruses, Carlaviruses or Vitiviruses.
